(19) United States
(12) Patent Application Publication
(10) Pub. No.: US 2004/0156794 A1
(43) Pub. Date: Aug. 12, 2004

(54) Bioerodible and bioadhesive confectionery products and methods of making same

(76) Inventors: David G. Barkalow, Deerfield, IL (US); Robert B. Friedman, Chicago, IL (US); Elena S. Mirzoeva, Cary, IL (US); Mark A. Deters, Joliet, IL (US)

Correspondence Address: BELL, BOYD & LLOYD LLC, P. O. BOX 1135, CHICAGO, IL, 60690-1135, US

(21) Appl. No.: 10/364,961

(22) Filed: Feb. 11, 2003

(51) Int. Cl. A61K 9/68

(52) U.S. Cl. 424/48

(57) ABSTRACT

The present invention relates generally to confectionery products. The present invention provides both bioadhesive and bioerodible confectionery formulations and methods of making and using the same; more specifically, the present invention provides delivery systems to release flavors, sweeteners, cooling agents, active ingredients and the like to consumer for an extended period of time. Further, these confectionery products may also provide a venue to mask off notes and bitter flavors of drugs or other active ingredients to consumers.

### BACKGROUND OF THE INVENTION

The present invention relates generally to confectionery products. More specifically, the present invention provides bioerodible and bioadhesive confectionery formulations and methods of making same.

A wide variety of confectionery products are known. These products can vary in taste, form, and shape. Confectionery products have been molded, extruded, or otherwise shaped into various forms over the years. For example, bubble gum has been formed into shapes such as flat sticks, cylinders, cubes, cigars, shredded chew, and the like. It is generally known that providing a confectionery product in a novelty form can enhance the marketability of a product.

Recently, edible films have emerged as a growing product in the marketplace. These products provide a discreet, fast-dissolving confectionery product with instant flavor release and breath freshening capabilities. To this end, edible films can provide flavor and/or oral care agents, e.g., breath freshening to the consumer. Such films typically include a film former and flavor or other ingredient. See, for example, U.S. Pat. No. 5,948,430 and U.S. Application Publication No. US2001/0022964 A1.

Edible film products are provided to the consumer in strip form. The strips are usually sized so that they can be placed on the tongue of a consumer. In this regard, the edible film strips typically have a size of a postage stamp or slightly larger. These strips preferably have a supple texture and are non-self adhering.

One type of edible film product is distributed by the Wm. Wrigley Jr. Company under the brand name Eclipse® Flash Strips. Another type of edible film is distributed by Pfizer Consumer Healthcare under the name Listerine® PocketPaks™. Typically, these products are packaged in a plastic container that includes a top that can open along a hinge. A stack of strips are located in an interior of the package one on top of another. The package is designed so that the consumer can open the container and remove one strip from the stack with his or her finger.

Although edible films provide consumers with a unique confectionery experience, this experience itself is short lived. Generally, most edible films quickly adhere to and dissolve in the mouth of a consumer within 30 seconds of consumption.

Another type of consumable product is a group of products that are referred to as bioerodible. These products are made from slow dissolving hydrocolloids. These hydrocolloids can form loose bonds to various tissues in the oral cavity. A bioerodible composition can remain in the oral cavity, dissolving slowly, for up to 5 hours. However, because bioerodible compositions generally form loose or weak bonds with epithelial tissue or mucin, they do not fully adhere to surfaces and are movable within the oral mucosa by various interactions, e.g., movement of the tongue.

Similar to bioerodible products are another group of products that are referred to as bioadhesive. These products are made from water-soluble and/or water insoluble polymers. Due to their structure, these products readily adhere to epithelial tissue or to the mucous coating that exists on the tissue. They form stronger bonds with tissues and mucin and are difficult to move or remove. When the bioadhesive attaches to the mucous coating, the phenomenon is referred to as mucoadhesion. Bioadhesion can be modeled after bacterial attachment to tissue surfaces and mucoadhesion can be modeled after mucin organization on epithelial tissue.

The residence time of a bioadhesive product on tissue and mucin is controlled by the characteristics of the polymer. Particularly relevant is whether the polymer is soluble in water or not. Bioadhesives can adhere to various regions within the mouth of consumers and dissolve slowly; bioadhesives can remain in the oral for periods ranging from 1 hour up to 72 hours.

It is known to use bioadhesives in a drug delivery system. See e.g., U.S. Pat. Nos. 6,159,498 and 5,800,832. Further, bioadhesives are also known to act as a breath freshening devices. See, for example U.S. Pat. No. 5,700,478. Unfortunately, however, some of the prior applications of bioadhesive polymers are not food approved, have poor mouth feel, or simply cannot deliver complex flavor/sweetener/cooling systems.

### SUMMARY OF THE INVENTION

The present invention provides improved confectionery formulations and methods of making and using the same that include bioadhesive characteristics. These products can also include components having bioerodible characteristics. In part, the present invention provides delivery systems to release flavors, sweeteners, cooling agents, active ingredients and the like to consumer for an extended period of time. Further, these confectionery products may also provide a venue to mask off notes and bitter flavors of drugs or other active ingredients to consumers.

Due to the novel bioerodible and bioadhesive confectionery products for application to mucosal surfaces of the present invention, the consumer can be provided with unique oral effects. Some of the effects include, but are not limited to, long-lasting flavor, cooling or sweetness, flavor bursts, trigeminal effects, multiple flavors, malodor control, tooth whitening, enhanced flavor perception, and second party breath freshening.

Aside from adhering the epithelial tissue or the mucin which exists on epithelial tissues, at the same time, the bioadhesive or bioerodible compositions may adhere to each other or adhere to other backing substrates or layers such as confectionery products including, but limited to, edible films, chewing gums, chocolates, mints, glassy candies, and chewy candies, alone or in any combination, including, but not limited to, hard candies such as peppermint sticks, fruit drops, and clear mints; soft candies such as marshmallows, jellies, nougats, chocolates, fudge, caramels, panned candies, gels, chewing gums, pastas, chewy candies, pressed mints, and edible films.

Bioerodible products and bioadhesive/mucoadhesive products can be “stand-alone” products, or may be combined to provide an opportunity to deliver steady, long-lasting oral sensations, active ingredients, or breath freshening capabilities over a given period of time. Further, these products can be tailored to adhere to specific oral surfaces such as mucin, teeth, dental apparatuses, and oral tissues. Bioadhesive and bioerodible compositions can also adhere to a backing layer or substrate, such as confectionery products including but not limiting to hard boiled candies, mints, chocolates, edible films, chewing gums, and chewy candies for a unique confectionery experience.

To this end, a variety of edible improved bioadhesive confectionery products are provided. The bioadhesive product can comprise a water-soluble polymer, a water-insoluble polymer or combination of the two.

In an embodiment, the bioadhesive product is designed to adhere to a specific region of the oral cavity. For example, the bioadhesive product can be designed to adhere to: the mucin of the consumer; the lining mucosa of the consumer; the masticatory mucosa of the consumer; the specialized mucosa of the consumer; the teeth of the consumer; the cheek of the consumer; the ventral tongue of the consumer; the gums surrounding the teeth of the consumer; and the roof of the mouth of the consumer.

In an embodiment, the bioadhesive product adheres to specific regions on the tongue to stimulate certain tastes. For example, the bioadhesive can comprise citric acid and be placed on the either side of the tongue of the mouth of a consumer to stimulate the taste buds of the consumer for the consumer to perceive sourness.

The bioadhesive product can be designed so that the bioadhesive remains attached to a surface in the oral cavity for a variety of time periods. For example, products can be designed that adhere to a surface of the oral cavity for at least 5 minutes; at least 1 hour; and up to 10 hours.

In an embodiment, the bioadhesive product comprises at least one water-soluble polymer chosen from the group consisting of agar, alginic acid, alginate, amylose, high amylose starch, gum arabic, carrageenan, processed euchema seaweed, casein, carboxymethyl cellulose (CMC), carboxyvinyl copolymer, hydroxypropylcellulose (HPC), hydroxypropylmethylcellulose (HPMC), hydroxyethylcellulose (HEC), methyl cellulose, microcrystalline cellulose (MCC), sodium carboxymethyl cellulose, natural celluloses, chitin, chitosan, collagen, dextran, polydextran, elsinan, gelatin, gellan gum, guar gum, gelatin, ghatti gum, karaya gum, gluten, konjac, levan, locust bean gum, maltodextrin, methylmethacrylate copolymer, oat gum, pectin, low methoxy pectin, polyethylene glycol, polylysine, polybrene, polyvinylpyrrolidone, polyvinyl alcohol, polyacrylic acid, propylene glycol, protein, pullulan, starch, modified starches, soy protein, tara gum, tamarind gum, tragacanth, whey protein, xanthan gum, and zein. The water-soluble polymer can be present in an amount of between 1% to about 60% by wet weight, or 15% to about 30% by wet weight of the final film composition.

In an embodiment of the present invention, the bioadhesive product comprises at least one water-insoluble polymer chosen from the group consisting of carbopol, curdlan, polycarbophil, cross-linked polymethacrylic acid, high molecular weight celluloses such as hydroxypropylmethylcellulose, polymethylmethacrylate, and ethyl cellulose.

In an embodiment, the bioadhesive product comprises at least one of the above-identified water-soluble polymers and at least one of the above-identified water-insoluble polymers.

The bioadhesive product can include and release as it dissolves in the mouth of the consumer a medicament. In an embodiment, the medicament is chosen from the group consisting of pH control agent, tartar control, malodor control, breath freshening agents, caries control, enzymes, anti-plaque/anti-gingivitis agents, remineralization/deminerliazation agents, saliva stimulating agents, energizing agents, pharmaceutical agents, nutraceutical agents, vitamins, minerals, and combinations thereof.

The bioadhesive product can be designed to release a flavor as it dissolves. In an embodiment, the flavor is chosen from the group consisting of essential oils, synthetic flavors and mixtures including, but not limited to, oils derived from plants and fruits such as citrus oil, fruit essences, peppermint oil, spearmint oil, other mint oils, oil of wintergreen, and anise, flavors derived from spices and the like, flavor oils with germ killing properties such as menthol, eucalyptol, thymol, methyl salicylate, licorice, cinnamic aldehyde, like flavoring agents or combinations thereof.

In a still further embodiment of the present invention, the bioadhesive product comprises more than one layer. By providing multilayer products, unique products having a variety of characteristics can be provided. For example, in an embodiment, the bioadhesive product comprises three layers. In an example of such a product, the first layer is an edible film, the second layer is a bioadhesive, and the third layer is an edible film. In another embodiment, the bioadhesive product has three layers consisting of an edible film, a bioadhesive, and a bioerodible disc.

A variety of bioadhesive products are possible. For example, a bioadhesive product can be provided including sides that are not parallel. The bioadhesive can be shaped similar to the area it will adhere to. The bioadhesive layer can include a surface that is textured. The bioadhesive layer can include a surface that has suction cups.

In an embodiment, at least one surface of the bioadhesive product has a design.

In an embodiment, the bioadhesive includes an ingredient that creates an oral sensation as the product remains adhered to a region on the oral cavity in the mouth of a consumer. In another embodiment, upon dissolution of the bioadhesive, the bioadhesive releases mucoadhesive micro-beads that adhere to mucosal surfaces for longer lasting oral sensations.

In an embodiment of the present invention, a bioadhesive is provided comprising a body having a shape that is indicative of the site of adherence of the bioadhesive. For example, a tooth-shaped bioadhesive product is provided that is designed to adhere to the teeth of the consumer.

In an embodiment, the bioadhesive is attached to a backing. For example, the backing can be a bioerodible composition. In a further embodiment, the backing comprises a confectionery product chosen from the group consisting of chewing gums, edible films, chewy candies, mints, chocolates, gels, soft candies, and hard-boiled candies. The backing can be a different color than the bioadhesive product.

In addition to having a bioadhesive component, the bioadhesive products can include a bioerodible component. The bioerodible component can be provided in combination with the bioadhesive component and with other components, e.g., a thin film. In an embodiment the bioerodible product comprises a water-soluble polymer chosen from the group consisting of agar, alginic acid, alginate, amylose, high amylose starch, gum arabic, carrageenan, processed euchema seaweed, casein, carboxymethyl cellulose (CMC), carboxyvinyl copolymer, hydroxypropylcellulose (HPC), hydroxypropylmethylcellulose (HPMC), hydroxyethylcellulose (HEC), methyl cellulose, microcrystalline cellulose (MCC), sodium carboxymethyl cellulose, natural celluloses, chitin, chitosan, collagen, curdlan, dextran, polydextran, elsinan, gelatin, gellan gum, guar gum, ghatti gum, karaya gum, gluten, konjac, levan, locust bean gum, maltodextrin, methylmethacrylate copolymer, oat gum, pectin, low methoxy pectin, polyethylene glycol, polylysine, polybrene, polyvinylpyrrolidone, polyvinyl alcohol, polyacrylic acid, propylene glycol, protein, pullulan, starch, modified starches, soy protein, tara gum, tamarind gum, tragacanth, whey protein, xanthan gum, and zein. Preferably, the water-soluble polymer comprises between 1% to about 60% by wet weight, or 10% to about 20% by wet weight of the bioerodible composition.

In an embodiment, the bioerodible product comprises at least one slow-dissolving hydrocolloid chosen from the group consisting of hydroxypropylcellulose, hydroxypropylmethylcellulose, ethyl cellulose, soy protein, casein, gelatin, and high molecular weight polysaccharides such as alginates, carrageenan, guar gum, tragacanth gum, gum arabic, and carboxymethylcellulose. If desired, the bioerodible product can contain at least one additional hydrocolloid to reduce the brittleness of the slow-dissolving hydrocolloids.

If desired, the bioerodible portion of the product can also include a medicament. Again, the medicament can be chosen from the group consisting of pH control agent, tartar control, malodor control, breath freshening agents, caries control, enzymes, anti-plaque/anti-gingivitis agents, saliva stimulating agents, energizing agents, pharmaceutical agents, nutraceutical agents, botanicals, vitamins, minerals, other medicaments or combinations thereof.

In an embodiment, the bioerodible portion includes a flavor chosen from the group consisting of essential oils, synthetic flavors and mixtures including, but not limited to, oils derived from plants and fruits such as citrus oil, fruit essences, peppermint oil, spearmint oil, other mint oils, oil of wintergreen, anise, flavors derived from spices and the like, flavor oils with germ killing properties such as menthol, eucalyptol, thymol, methyl salicylate, licorice, cinnamic aldehyde, like flavoring agents or combinations thereof.

In an embodiment of the present invention, the bioerodible product can include a backing, e.g., an edible film. In an embodiment, the bioerodible product has a different color than the backing layer.

In an embodiment, a product is provided comprising a bioerodible part and a bioadhesive part, the bioerodible part has a different color than the bioadhesive part.

In an embodiment, the bioerodible product dissolves in the mouth of the consumer after 30 seconds; in embodiment, the bioerodible product dissolves in the mouth of the consumer after one minute.

It is an advantage of the present invention to provide improved products having bioadhesive properties.

Another advantage of the present invention is to provide novel forms of confectionery products.

Moreover, an advantage of the present invention is to provide products that utilize at least some of the characteristics of thin film, bioerodible, and bioadhesive products.

Further, an advantage of the present invention is to provide bioadhesive products that can be used with foods and beverages.

Still an advantage of the present invention is to provide methods for producing bioadhesive products.

And another advantage of the present invention is to provide bioadhesive confectionery products.

Furthermore, an advantage of the present invention is to provide improved methods of generating revenue from confectionery products.

Moreover, an advantage of the present invention is to provide improved methods for developing products between two or more entities.

Additional features and advantages of the present invention are described in, and will be apparent in, the detailed description of the presently preferred embodiments.

### BRIEF DESCRIPTION OF THE FIGURES

FIG. 1 illustrates a perspective view of an embodiment of a product of the present invention.

FIG. 2 illustrates a cross-sectional view of the product of FIG. 1 taken along lines II-II.

### DETAILED DESCRIPTION OF THE INVENTION

The present invention provides edible bioadhesive products and methods of using and making the same. As noted above, bioadhesives can be used for delivery of products via mucosal epithelial tissue. Since bioadhesives avail the consumer to increased residence times, this offers improvements in local delivery to specific oral regions.

As used herein, the term “bioadhesive” is defined as any substance that can adhere to a biological substrate and is capable of being retained on that surface for an extended period of time. An “extended period of time” is defined herein as being at least one minute. Thus, in order to meet the term bioadhesive product, at least a portion of the product must be capable of adhering to a biological surface, e.g., mucous membrane, for at least one minute.

Bioadhesives can adhere to different substrates; however, bioadhesives generally adhere to membranes or the mucin layer of those membranes. Generally, these products take the appearance of films or discs which fit both easily and comfortably in the mouth of a consumer.

Bioadhesive products can be made from water-soluble and/or water insoluble polymers that readily adhere to epithelial tissue or to the mucous coating that exists on the tissue. Exactly how long the bioadhesive/mucoadhesive adheres to such surfaces is dependent upon the characteristics of the substance(s) incorporated into the bioadhesive. In particular to the oral cavity, the residence time is dependent upon the water-solubility of the bioadhesive being used as well as the molecular weight of the polymer being employed. Generally, polymers having molecular weights greater than 100,000 are capable of inducing good wet adhesion properties.

Other factors can contribute to the residence time of the bioadhesive, particularly the physiochemical characteristics of the bioadhesive (water dispersibility, degree of hydration, ionization of hydrocolloids, rate and capacity of water consumption), and optionally, the backing substrate/composition, and any other actives or ingredients that is incorporated into the complex. Suitable polymers that can be used to form bioadhesives include soluble and insoluble, nonbiodegradable and biodegradable polymers. These can be hydrogels or thermoplastics, homopolymers, copolymers or blends, natural or synthetic.

The oral cavity can be divided into three distinct functional regions: the lining mucosa (buccal, sublingual, and soft palate); the masticatory mucosa (hard palate and gingival); and the specialized mucosa (dorsal tongue). Each region has unique characteristics such as thickness and keratinization, therefore making the permeability of an active, flavor, sweetener, etc., and the adherence of a bioadhesive, differ from each region. In general, bioadhesives can adhere to the cheek (buccal tissue), the floor of the mouth (sublingual tissue), the gums surrounding teeth (gingival tissue), the “roof” of the mouth (palatal tissue), and the teeth.

Generally, bioadhesives can remain in the oral cavity for any desired length of time. Some bioadhesives may remain attached to epithelial tissue for 5 minutes or even up to 72 hours. The length of time the bioadhesive remains attached to particular substrate heavily correlates to the water solubility of the bioadhesive being employed. Water solubility is often dependent on the number and type of cross-links that occur between polymer chains. However, lightly cross-linked and hence water swellable but, still water-insoluble polymers are preferred as mucoadhesives given that they are not easily removed from the site of application.

For a shorter bioadhesive retention time and weaker site adhesion properties, any suitable water-soluble polymer may be used. Suitable water-soluble bioadhesive polymers include, but are not limited to, agar, alginic acid, alginate, amylose, high amylose starch, gum arabic, carrageenan, processed euchema seaweed, casein, carboxymethyl cellulose (CMC), carboxyvinyl copolymer, hydroxypropylcellulose (HPC), hydroxypropylmethylcellulose (HPMC), hydroxyethylcellulose (HEC), methyl cellulose, microcrystalline cellulose (MCC), sodium carboxymethyl cellulose, natural celluloses, chitin, chitosan, collagen, dextran, polydextran, elsinan, gellan gum, guar gum, ghatti gum, karaya gum, gluten, konjac, levan, locust bean gum, maltodextrin, methylmethacrylate copolymer, oat gum, pectin, low methoxy pectin, polyethylene glycol, polylysine, polybrene, polyvinylpyrrolidone, polyvinyl alcohol, polyacrylic acid, propylene glycol, protein, pullulan, starch, modified starches, soy protein, tara gum, tamarind gum, tragacanth, whey protein, xanthan gum, and zein. These water-soluble polymers can comprise between approximately 1% to about approximately 60% by wet weight, or approximately 15% to about approximately 30% by wet weight of the bioadhesive composition.

For longer bioadhesion and stronger bonds between surfaces any suitable water-insoluble polymer may be used. Suitable water-insoluble polymers include but are not limited to carbopol, curdlan, polycarbophil, polymethacrylic acid, hydroxypropylmethylcellulose (HPMC), polymethylmethacrylate, and ethyl cellulose. The water-insoluble polymers can comprise between approximately 1% to about 60% by wet weight, or approximately 15% to about 30% by wet weight of the bioadhesive composition.

Other potential components of bioadhesion may include materials such as zeolites, silica gels, mineral and natural fillers (i.e. cotton, wood), rosin esters, mineral salts such as zinc or calcium, corn syrup, hard candies, food grade acids, glycerin and enzymes.

As noted above, bioerodible products can also be combined with other components to create a bioadhesive product, i.e., a product that can adhere for at least one minute. Bioerodible compositions often take the shape of an edible film or disc, and are comprised of polymers. Bioerodible products are water-soluble polymer chosen from the group consisting of agar, alginic acid, alginate, amylose, high amylose starch, gum arabic, carrageenan, processed euchema seaweed, casein, carboxymethyl cellulose (CMC), carboxyvinyl copolymer, hydroxypropylcellulose (HPC), hydroxypropylmethylcellulose (HPMC), hydroxyethylcellulose (HEC), methyl cellulose, microcrystalline cellulose (MCC), sodium carboxymethyl cellulose, natural celluloses, chitin, chitosan, collagen, curdlan, dextran, polydextran, elsinan, gelatin, gellan gum, guar gum, ghatti gum, karaya gum, gluten, konjac, levan, locust bean gum, maltodextrin, methylmethacrylate copolymer, oat gum, pectin, low methoxy pectin, polyethylene glycol, polylysine, polybrene, polyvinylpyrrolidone, polyvinyl alcohol, polyacrylic acid, propylene glycol, protein, pullulan, starch, modified starches, soy protein, tara gum, tamarind gum, tragacanth, whey protein, xanthan gum, and zein. These products comprise between approximately 1% to about 60% by wet weight of water-soluble polymers, or approximately 10% to about 20% by wet weight of water-soluble polymers of the bioerodible composition.

Bioerodible products can also comprise a slow-dissolving hydrocolloid chosen from the group consisting of hydroxypropylcellulose, hydroxypropylmethylcellulose, ethyl cellulose, soy protein, casein, gelatin, high molecular weight polysaccharides such as alginates, carrageenan, guar gum, tragacanth gum, gum arabic, and carboxymethylcellulose. Specifically, between 1% to about 60% by wet weight of the bioerodible composition can be hydrocolloid. The bioerodible product may also contain at least one additional hydrocolloid to reduce brittleness of the slow-dissolving hydrocolloids.

With respect to either the bioadhesive or bioerodible composition, they can also contain a certain amount of plasticizing agents. This will assist in maintaining the flexibility and reduce the brittleness of the bioadhesive. Suitable plasticizers or softeners may include but are not limited to tallow, hydrogenated tallow, hydrogenated and partially hydrogenated vegetable oils, cocoa butter, polyalcohols such as sorbitol, glycerol, maltitol, mannitol, and erythritol, polyethylene glycol, propylene glycol, invert sugars, corn syrup, lecithin, hydrogenated lecithin, enzyme-modified lecithin, mono-, di-, and triglycerides, acetylated monoglycerides, vitamin E, medium chain triglycerides such as coconut oil and fatty acids (e.g. stearic, palmitic, oleic, and linoleic acids), and combinations thereof. In an embodiment, the softener can constitute 0% to about 20% by wet weight, or approximately 0.01% to about 10% by wet weight of the bioadhesive or the bioerodible composition.

Fillers can also be incorporated into the bioadhesive or bioerodible compositions. Suitable food grade fillers include, but are not limited to, microcrystalline cellulose, cellulose polymers, such as wood, magnesium and calcium carbonate, ground limestone, silicates such as magnesium and aluminum silicate, clay, talc, titanium dioxide, monocalcium phosphate, di-calcium phosphate, tri-calcium phosphate, other bulk fillers or combinations thereof. The filler can comprise approximately 0.01% to about 30%, or approximately 1% to about 10% by wet weight of the bioadhesive or biocrodible composition.

Any suitable amount of sweetening agents may also be employed in the products of the present invention. Sugar sweeteners generally include saccharide-containing components including, but not limited to, sucrose, dextrose, maltose, dextrin, invert sugar, fructose, levulose, galactose, corn syrup solids, and the like, alone or in any combination. Sugarless sweeteners include, but are not limited to, sugar alcohols such as sorbitol, mannitol, xylitol, isomalt, hydrogenated starch hydrolysates, maltitol, and the like, alone or in any combination.

High intensity artificial sweeteners are preferably used, alone or in combination with the above. Preferred sweeteners include, but are not limited to, sucralose, aspartame, N-substituted APM derivatives such as neotame, salts of acesulfame, alitame, saccharin and its salts, cyclamic acid and its salts, glycyrrhizin, sucralose, dihydrochalcones such as neohesperidine, thaumatin, monellin, and the like, alone or in any combination. In order to provide enhanced or delayed sweetness, or to provide texture to, it may be desirable to encapsulate the sweetener. Such techniques as wet granulation, wax granulation, spray drying, spray chilling, fluid bed coating, coacervation, and fiber extension may be used to achieve the desired characteristics.

Combinations of sugar and/or sugarless sweeteners may be used in the film product. Additionally, a softening agent may also provide additional sweetness such as with aqueous sugar or alditol solutions. The incorporation of some sweeteners may also provide health benefits to the consumer.

Cooling agents may also be employed in the present invention, cooling agents include, but are not limited to, menthol, p-mentha-3, 8-diol, 1-isopulegol, menthone ketals, menthyl lactate, monomenthol succinate, menthyl pyrrolidone carboxylate, 3-1-menthoxypropane-1, 2-diol (Takasago Cooling Agent, TCA), N-ethyl-p-menthane-3-carboxamide (WS-3), 2-isopropyl-N, 2,3-trimethylbutyramide (WS-23), other cyclic and acyclic carboxamides and 3,3,5-trimethylcyclohexanol. It will often be desirable to include two or more of these cooling agents in combination. Cooling agents also impart the perception of breath freshening to the product.

Again, these cooling agents may be encapsulated or spray dried and added to the compositions, e.g., bioadhesive products. Such cooling agents may assist in flavor enhancement, taste-masking properties, or create various oral sensations.

A variety of flavoring agents can also be added to the bioadhesive or bioerodible compositions. Any suitable amount and type of artificial and/or natural flavoring agents can be used in any sensorially acceptable fashion. For example, the flavor can constitute approximately 0.01% to approximately 20% by wet weight of the bioadhesive/bioerodible composition, preferably about 4% to about 15%. The flavoring agent can include, for example, essential oils, synthetic flavors or mixtures including but not limited to oils derived from plants and fruits such as citrus oil, fruit essences, peppermint oil, spearmint oil, other mint oils, clove oils, oil of wintergreen, anise and the like, flavor oils with germ killing properties such as menthol, eucalyptol, methyl sclicylate, thymol, cinnamic aldehyde, licorice, like flavoring agents or combinations thereof. Fruit flavors such as lemon, lime, orange, grape, apple, peach, strawberry, banana, raspberry, cherry, kiwi, melon and mixed fruit can be employed. Spice flavors may also be included such as clove, cinnamon, and licorice. Of course, any desired blend of these flavors either within or across groups can be prepared.

Flavors can act as salivary stimulating agents, especially if used in conjunction with a food grade acid, as well as assisting in masking oral malodors, creating oral sensations and enhancing the aroma or smell of the product before or after consumption. Flavor crystals or beads can be employed to create texture, intensify flavor, or generate flavor bursts.

The flavor can be distributed evenly throughout the product by emulsification. Any suitable amount and type of natural and/or synthetic food-grade emulsifier can be used. For example, the emulsifier can include lecithin, food-grade non-ionic emulsifiers, such as fatty acids (C10-C18), mono and diacyl glycerides, ox bile extract, polyglycerol esters, polyethylene sorbitan esters, propylene glycol, sorbitan monopalmitate, sorbitan tristerate, other like emulsifiers or combinations thereof.

Tartness may be employed to enhance many fruit flavors and can be provided by incorporating food grade acids into the composition of the present invention. Examples of food acid suitable for use in the present invention include citric, tartaric, malic, fumaric, and malic acids. It may be necessary to encapsulate the acids using any of several known processes to prevent reactions with colors, flavors, polymers, sweeteners, and other components.

The bioadhesive or bioerodible formulations of the present invention can also include colorants or coloring agents that can be used in any suitable amount to produce a desired color. Further, the products of the present invention may have colored stripes and/or other related designs or shapes to produce color contrasts on the bioadhesive products. Particularly, if a backing layer is also present, designs and colors may assist the consumer in properly placing the bioadhesive surface to an epithelial tissue. Coloring agents can include, for example, natural food colors and dyes suitable for food, drug, and cosmetic applications. The colorants are typically known as FD&C dyes and lakes.

A variety of other suitable ingredients can be added to the compositions of the present invention. For example, any suitable medicament for oral cleansing, breath freshening or the like can be added to the bioadhesive formulation. The medicaments can include, for example, pH control agents, such as urea and buffers; inorganic components for tartar caries control such as phosphates, pyrophosphates, polyphosphates and fluorides; enzymes such as proteolytic and glycolydic enzymes to disrupt plaque and the pellicle, enzymes such as oxidoreductase to oxidize stains on teeth and enzymes which react with sulfur compounds in breath. (Such enzymes are produced by Genencor, Enzyme Development Corporation, Valley Research and Novozymes); breath freshening agents, such as food acceptable metal salts such as copper or zinc gluconate; anti-plaque/anti-gingivitis agents, such as chlorohexidene, CPC, and triclosan; remineralizing or deminerlizing agents; saliva stimulating agents including, for example, food acids such as citric, lactic, malic, succinic, ascorbic, adipic, fumaric, and tartaric acids; pharmaceutical agents, nutraceutical agents, vitamins, mineral, other like medicaments or combinations thereof.

Other potential active ingredients may include but are not limited to botanicals and functional ingredients such as coenzyme Q10, pro-, pre-, and macrobiotics, proanthocyanins, St. John's Wart, red clover, black cohosh, milk thistle, lutein, lycopene, fenugreek, echinacea, glucosamine, oxomate, horse chestnut seed extract, soy extracts, and other like active ingredients alone or in any combination.

Specific vitamins and minerals which are especially suited to delivery by orally consumed bioadhesives include vitamins B12, B1, B2, B6, A, D, E, folic acid, selenium, iron, and zinc.

Depending on the ingredients being used to make the bioadhesive product, preservatives may also be employed to ensure the safety and quality of the final composition. Suitable preservatives include but are not limited sorbic acid, sodium benzoate, potassium sorbate, methyl p-hydroxybenzoate, sodium propionate, and propyl p-hydroxybenzoate alone or in any combination.

The bioadhesive compositions can be “stand-alone” confectionery products. Optionally, the bioadhesive and bioerodile composition may also be attached or anchored to each other; or still further, they may individually or together adhere to what is called a “backing”. This backing may consist of a variety of ingredients or food products. Preferably, the backing consists of but is not limited to confectionery compositions including but not limiting to edible films, hard candies, chewy candies, jellies, soft candies, pressed candies, tablets, gels, chocolates, syrups, and pastes, alone or in any combination.

Further, the bioadhesive product may be shaped into any desired shape. For example the shape of the bioadhesive may be in a functional shape, such as the shape of the letter “U” to better fit on the floor of the mouth underneath the tongue. Any cross-sectional shape may be used, including by way of example and not limitation, triangle, oval, circular, and hexagonal shapes. The shape of the bioadhesive may be fanciful if desired or representative of any other person, object, character, or any other design desired.

Neither composition is limited to a particular size or thickness. However, the thickness of the composition would impact the length of retention while in the oral cavity and control the release of actives, flavors, sweeteners, cooling agents, etc., employed. Further, one means of controlling release and providing a steady flow of ingredients and tastes to the consumer is by making a multi-layered bioadhesive or bioerodible product.

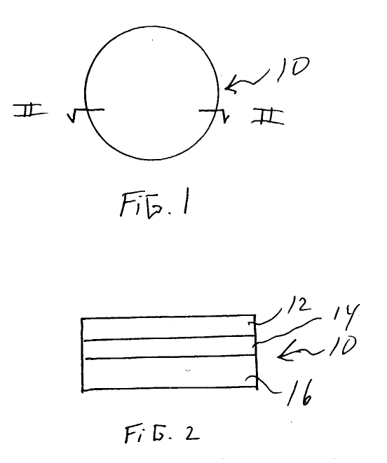

Referring now to the drawings and specifically FIG. 1, a bioadhesive product 10 is illustrated. Although the product is illustrated as having a circular shape, as noted above, a variety of shapes can be provided; again, what is important is that the product have bioadhesive properties. As noted above, as used in the application, bioadhesive properties means that at least a portion of the product will adhere to a biological surface, e.g., a portion of the oral cavity for at least one minute.

Referring now to FIG. 2, as illustrated, the product includes at least three layers 12, 14, and 16. In the illustrated embodiment, the first layer 12 is a bioadhesive product.

The second layer 14 is comprised of a material having bioerodible properties. Compositions having this property are discussed above.

The third layer 16 is comprised of an edible thin film. Compositions having this property are discussed above.

This thin film product can be a variety of edible thin film products. In this regard, reference is made to U.S. patent application Ser. No. 10/228,742 filed Aug. 27, 2002 entitled “Rolled Edible Thin Film Products and Methods of Making Same,” the entire disclosure of which is incorporated herein.

Briefly, that application discloses a variety of forms of consumable edible thin films. As used in that application, and as used herein, the term “edible thin film” refers to compositions that include a film former substrate and are designed to adhere to at least a portion of the oral cavity of the consumer and rapidly dissolve therein. “Rapidly dissolve” means that the substrate dissolves in less than 20 seconds, preferably less than 15 seconds, and most preferably less than 10 seconds. To dissolve means to substantially lose the shape and form of the substrate. Examples of edible thin film products are the Listerine® PocketPaks™ oral care strips sold by Pfizer and the Eclipse® Flash Strips sold by Wrigley.

Thus, the product 10 is designed so that the first layer 12, when it comes into contact with a portion of the oral cavity of the consumer, will adhere thereto. Therefore, in use, the consumer will place the product 10 in his or her mouth. The bioadhesive layer 12 will adhere readily to a region within the oral cavity and slowly begin dissolving. During this time, the thin film layer 16 will dissolve almost immediately while coming into contact with the consumer's natural salivary flow and enzymes. Once the thin film layer 16 dissolves, the bioerodible layer 14 will begin to erode in the mouth of the consumer due to salivary hydration and enzymatic mechanisms. Once layers 14 and 16 have dissolved, layer 12 will likely remain and continue to degrade until dissolved.

For example, a wide variety of confectionery products can be envisioned for providing flavors or oral sensations to the consumer. Further, the product can be used to deliver active ingredients such as medicaments.

It should be appreciated that the number of layers and the order of layers can be varied. Therefore, products having more than one biocrodible, bioadhesive, and thin film strip layer are envisioned. The only limitation is that at least one of the layers must provide bioadhesive characteristics, allowing the product to function as a bioadhesive product.

These “layers” are interchangeable and any desired thickness for each particular layer may be incorporated. For example, in an embodiment of the product 10, the first layer 12 can be a bioadhesive that is approximately 0.05 mm thick, the second layer 14 is an edible film that is approximately 0.03 mm, and the third layer 16 is a bioerodible composition that is approximately 0.10 mm thick. Potentially, each of these layers can reach dissolution at different times, have different colors, and have different flavors. Further, thickness of any of the layers incorporated can be used to control the time of dissolution.

In addition, texture may be added to the bioadhesive product 10 for a variety of reasons. Texture may increase the bioadhesive/mucoadhesive surface area thereby creating a larger area of adhesion and increase uptake of desired components. Also, the bioadhesive product 10 may be textured to increase adhesion strength or possibly create the actual source of adhesion. Other texturizers may simply create mouth feel and other oral sensations, or simply make the bioadhesive visually appealing.

It should be appreciated that any suitable type, number and arrangement of process steps or procedures (e.g. mixing, heating, drying, cooling, addition of ingredients), process parameters (e.g. temperature, pressure, pH, humidity, process times), or the like can be utilized to practice the present invention.

Of course, a variety of package/container designs are possible. For example, the products may be available in a multiple layered package, wherein the primary package is used to dispense the bioadhesive and is combined with a secondary package that may be used to carry, protect, and be refilled with the primary package. Also, the primary package may have three separate compartments containing three primary packages having a bioadhesive film, a bioerodible disc, and an edible film, respectively, in each separate container. Suitable packaging materials include, but are not limited to, plastic overwrap, metal tins, blister packs, vacuum formed plastic mold, injection molded plastic or corrugated paper board. The packaging materials selected may assist in maintaining the integrity of the product during shelf life depending on the components of the product. However, it should be noted that any container design could be used as long as it allows the consumer to access the product.

The present invention also provides a variety of marketing opportunities and methods of conducting a business. For example, the products allow improved methods for generating revenue from marketing products such as those set forth herein.

In addition, the present invention provides unique opportunities for at least two entities to work together to design products. For example, a pharmaceutical company having an expertise in drug delivery and/or bioadhesives can work with a company having an expertise in flavor and/or confectionery products, e.g., chewing gum, to design more suitable drug delivery products. Of course, other entities, aside from two companies, can work together to develop a product, e.g., a company and a university. These entities can also work together to design unique confectionery products. For example, products can be developed that utilize a flavor that is distinctive of a confectionery product that had been marketed prior to development of the bioadhesive product, e.g., flavors that have been used in chewing gums such as Juicy Fruit®, Big Red®, Eclipse®, Doublemint®, and Dentyne®.

As used herein, working together can be via, for example, a contract, licensing agreement, or joint venture. Once the product is developed, each of the entities, e.g., companies, can either each sell the product or only one company may sell the product or, the companies can divide up the resultant products along product lines; the confectionery company selling confectionery product and the pharmaceutical company selling pharmaceutical type products.

By way of example and not limitation, the following examples illustrate various embodiments of the bioadhesive and bioerodible confectionery formulations of the present invention.

### EXAMPLES

### Bioadhesive Composition

| | (gram wt.) | | | | | |
|---|---|---|---|---|---|---|
| Ingredient | Ex. 1 | Ex. 2 | Ex. 3 | Ex. 4 | Ex. 5 | Ex. 6 |
| Water | 845.92 | 838.9 | 802.1 | 750.0 | 771.3 | 695.0 |
| Hydroxy- | 50.0 | 25.0 | — | — | — | — |
| ethyl- | | | | | | |
| cellulose | | | | | | |
| Hydroxy- | — | — | — | — | — | 100.00 |
| propyl- | | | | | | |
| cellulose- | | | | | | |
| Carrageenan | 50.0 | — | — | — | — | — |
| Carboxy- | — | 72.0 | — | 100.0 | — | — |
| methyl- | | | | | | |
| cellulose | | | | | | |
| Poly- | — | 60.0 | 15.0 | — | — | — |
| ethylene | | | | | | |
| glycol | | | | | | |
| Sodium | — | — | 180.0 | — | — | — |
| alginate | | | | | | |
| Carbophil | — | — | — | 125.98 | — | 100.0 |
| Gelatin | — | — | — | — | 220.0 | 75.0 |
| Menthol | — | — | — | 5.0 | — | — |
| Aluminum | — | — | — | — | — | 15.0 |
| silicate | | | | | | |
| Titanium | — | — | — | 10.0 | — | — |
| dioxide | | | | | | |
| Calcium | 5.0 | 1.5 | — | — | — | — |
| carbonate | | | | | | |
| Citric | 1.0 | 1.5 | — | — | 0.5 | — |
| acid | | | | | | |
| Adipic | — | — | — | — | 2.0 | — |
| Acid | | | | | | |
| Liquid | — | — | — | — | — | 10.0 |
| sorbitol | | | | | | |
| Hydro- | — | — | — | — | — | 3.0 |
| genated | | | | | | |
| lecithin | | | | | | |
| Peppermint | 1.0 | 0.5 | — | 1.0 | 0.8 | — |
| Spearmint | — | 0.5 | — | 1.0 | — | — |
| Winter- | — | — | 0.5 | 2.0 | — | 1.0 |
| green | | | | | | |
| Menthyl | — | — | — | 0.2 | 0.2 | 0.5 |
| Lactate | | | | | | |
| WS-3 | — | — | — | — | 1.0 | 0.5 |
| Sucralose | — | — | 2.0 | — | — | — |
| Encap- | — | — | — | 5.0 | 1.0 | — |
| sulated | | | | | | |
| Aspartame | | | | | | |
| Caffeine | — | — | — | — | 3.0 | — |
| Color | 0.08 | — | 0.4 | — | 0.2 | — |
| Total Wt. | 1000.0 | 1000.0 | 1000.0 | 1000.0 | 1000.0 | 1000.0 |
| (grams) | | | | | | |

### EXAMPLES

### Bioadhesive Composition

| | (gram wt.) | | | | | |
|---|---|---|---|---|---|---|
| Ingredients | Ex. 7 | Ex. 8 | Ex. 9 | Ex. 10 | Ex. 11 | Ex. 12 |
| Water | 873.35 | 751.35 | 778.95 | 675.0 | 689.42 | 700.0 |
| Sodium | — | 200.0 | — | — | 50.0 | — |
| Alginate | | | | | | |
| Sodium | — | 5.0 | — | 75.0 | — | — |
| carboxy- | | | | | | |
| methyl- | | | | | | |
| cellulose | | | | | | |
| Karaya | 100.00 | 5.0 | — | — | 50.0 | 10.0 |
| gum | | | | | | |
| Methocel | — | — | 50.0 | — | 25.0 | — |
| (10 cps)* | | | | | | |
| Konjac | — | — | — | 50.0 | — | 5.0 |
| Tragacanth | — | — | 100.0 | — | 75.0 | 85.0 |
| Vitamin E | 15.0 | — | — | — | — | 100.0 |
| Linoleic | — | 10.0 | 0.50 | — | — | — |
| acid | | | | | | |
| Hydro- | — | — | — | 155.0 | 5.0 | — |
| genated | | | | | | |
| vegetable | | | | | | |
| oil | | | | | | |
| Cocoa | — | 10.0 | — | — | 53.0 | — |
| butter | | | | | | |
| Magnesium | — | 2.0 | — | — | — | 15.0 |
| silicate | | | | | | |
| Tricalcium | 5.0 | — | 30.0 | — | — | — |
| phosphate | | | | | | |
| Ground | — | 0.05 | — | 2.0 | 5.0 | 43.0 |
| limestone | | | | | | |
| Cinnamon | — | 10.0 | — | — | — | — |
| flavor | | | | | | |
| Spray | 2.0 | — | 20.0 | — | — | — |
| dried lemon | | | | | | |
| flavor | | | | | | |
| Orange | — | — | — | 5.0 | 10.0 | — |
| flavor | | | | | | |
| Clove | — | 0.5 | — | — | 2.5 | 28.0 |
| flavor | | | | | | |
| Alitame | — | — | 10.0 | — | — | — |
| Neotame | 1.0 | — | 2.0 | 0.8 | — | 1.0 |
| Encap- | — | 2.5 | — | 1.0 | — | 5.0 |
| sulated | | | | | | |
| acesulfame | | | | | | |
| k | | | | | | |
| Saccharin | — | — | — | — | 15.0 | — |
| Malic acid | — | — | 5.0 | 16.0 | — | — |
| Encap- | 3.0 | — | — | 4.0 | — | — |
| sulated | | | | | | |
| citric | | | | | | |
| acid | | | | | | |
| Glycerin | 0.5 | — | 3.0 | 15.0 | — | — |
| Menthol | — | 1.0 | 0.55 | — | — | 7.0 |
| WS-23 | 0.05 | — | — | — | 20.0 | — |
| WS-3 | 0.05 | — | — | 0.20 | — | 1.0 |
| Menthone | — | 2.5 | — | 0.50 | — | — |
| glycerol | | | | | | |
| ketal | | | | | | |
| Color | 0.0.5 | 0.10 | — | 0.50 | 0.08 | — |
| Total Wt. | 1000.0 | 1000.0 | 1000.0 | 1000.0 | 1000.0 | 1000.0 |
| (grams) | | | | | | |

*Methocel is a methylcellulose made by Dow Chemical.

### Examples

### Bioerodible Composition

| | (gram wt.) | | | | | |
|---|---|---|---|---|---|---|
| Ingredients | Ex. 13 | Ex. 14 | Ex. 15 | Ex. 16 | Ex. 17 | Ex. 18 |
| Water | 750.0 | 802.0 | 705.00 | 780.0 | 800.0 | 865.5 |
| Hydroxy- | 75.0 | 104.0 | — | — | — | 5.0 |
| ethyl- | | | | | | |
| cellulose | | | | | | |
| Hydroxy- | 75.0 | — | 52.45 | — | — | — |
| propyl- | | | | | | |
| cellulose | | | | | | |
| Methocel | 50.0 | — | 125.0 | — | — | — |
| (15 cps)* | | | | | | |
| Carbopol** | — | 52.0 | — | — | — | 30.0 |
| Casein | — | — | — | — | — | 85.0 |
| Polycar- | — | — | 60.0 | — | 76.5 | — |
| bophil | | | | | | |
| Sodium | — | — | — | 200.0 | 105.0 | — |
| alginate | | | | | | |
| Maltitol | 20.0 | — | — | — | 1.5 | — |
| Hydrogenated | — | 3.5 | — | — | — | — |
| tallow | | | | | | |
| Propylene | — | 4.5 | — | — | — | 5.0 |
| glycol | | | | | | |
| Oleic acid | — | — | 15.0 | — | — | — |
| Micro- | — | 6.2 | — | 4.4 | — | 1.0 |
| crystalline | | | | | | |
| cellulose | | | | | | |
| Talc | 0.2 | — | — | — | 1.0 | 0.4 |
| Di-calcium | — | — | 5.0 | — | — | — |
| phosphate | | | | | | |
| Magnesium | — | 1.5 | — | — | — | 0.6 |
| carbonate | | | | | | |
| Cherry | — | 3.0 | — | — | — | 3.5 |
| flavor | | | | | | |
| Encap- | — | 12.0 | — | — | — | — |
| sulated | | | | | | |
| peach | | | | | | |
| flavor | | | | | | |
| Green | — | — | — | 6.0 | — | — |
| apple | | | | | | |
| flavor | | | | | | |
| Methyl | 22.6 | — | 12.0 | — | — | — |
| salicylate | | | | | | |
| Licorice | — | — | 3.5 | — | 8.5 | — |
| flavor | | | | | | |
| Encap- | — | 5.42 | — | 0.5 | — | 1.0 |
| sulated | | | | | | |
| fumaric | | | | | | |
| acid | | | | | | |
| Citric acid | 0.2 | 1.0 | — | 0.5 | — | — |
| Monomenthol | — | 0.8 | — | — | 1.0 | — |
| succinate | | | | | | |
| Menthol | — | — | 12.0 | — | 1.0 | — |
| TCA | 5.0 | — | 1.5 | 0.5 | 1.0 | — |
| Aspartame | 1.0 | — | — | 8.0 | — | — |
| Acesul- | — | 2.5 | — | — | — | 1.6 |
| fame k | | | | | | |
| Alitame | — | 1.5 | — | — | 3.5 | — |
| Neotame | 0.1 | — | 5.0 | — | 1.0 | 0.8 |
| Encap- | 0.1 | — | 3.5 | — | — | 0.5 |
| sulated | | | | | | |
| sucralose | | | | | | |
| Color | 0.8 | 0.08 | 0.05 | 0.1 | — | 0.1 |
| Total Wt. | 1000.0 | 1000.0 | 1000.0 | 1000.0 | 1000.0 | 1000.0 |
| (grams) | | | | | | |

*Methocel is a methylcellulose from Dow Chemical.
**Carbopol is a high molecular weight copolymer of acrylic acid from Novcon, Inc.

### EXAMPLES

### Bioerodible Composition

| | (gram wt.) | | | | | |
|---|---|---|---|---|---|---|
| Ingredients | Ex. 19 | Ex. 20 | Ex. 21 | Ex. 22 | Ex. 23 | Ex. 24 |
| Water | 815.0 | 787.0 | 601.0 | 766.0 | 887.0 | 779.1 |
| Hydroxy- | — | 55.0 | — | 20.0 | — | 80.0 |
| ethyl- | | | | | | |
| cellulose | | | | | | |
| Hydroxy- | — | — | 150.0 | — | — | 110.0 |
| propyl- | | | | | | |
| methyl- | | | | | | |
| cellulose | | | | | | |
| Soy protein | 36.0 | — | — | — | — | — |
| Alginic | — | 50.0 | — | — | — | — |
| acid | | | | | | |
| Poly- | — | — | 145.0 | — | — | — |
| ethlyene | | | | | | |
| glycol | | | | | | |
| Tracaganth | 130.0 | — | — | — | — | — |
| Sodium | — | 55.0 | — | 191.5 | 100.92 | — |
| alginate | | | | | | |
| Carrageenan | 2.7 | — | — | — | — | — |
| Mannitol | — | 7.5 | 74.45 | — | — | — |
| Corn syrup | 10.0 | — | — | 3.5 | — | — |
| Lecithin | — | 1.5 | — | — | 4.5 | — |
| Neobee oil | — | — | 3.8 | — | — | — |
| Titanium | — | — | 1.2 | — | — | 0.5 |
| oxide | | | | | | |
| Palmitic | 3.0 | — | — | — | — | — |
| acid | | | | | | |
| Monocalcium | — | — | — | 1.0 | — | — |
| phosphate | | | | | | |
| Grape | 6.5 | — | — | — | 4.0 | — |
| flavor | | | | | | |
| Melon | 2.5 | — | — | — | — | — |
| flavor | | | | | | |
| Pineapple | — | — | 16.0 | — | — | — |
| flavor | | | | | | |
| Peppermint | — | 25.0 | — | 5.2 | — | — |
| oil | | | | | | |
| Spearmint | — | 10.0 | — | — | — | 28.0 |
| flavor | | | | | | |
| Encap- | — | — | 5.5 | — | 1.0 | — |
| sulated | | | | | | |
| tartaric | | | | | | |
| acid | | | | | | |
| Citric | 1.5 | — | — | — | 1.0 | 0.5 |
| acid | | | | | | |
| TCA | — | 3.0 | — | — | — | — |
| Menthyl | — | 1.5 | — | 1.5 | — | — |
| lactate | | | | | | |
| Eucalyptol | — | 1.0 | — | 5.5 | — | — |
| Sucralose | 1.0 | — | 2.0 | — | — | — |
| Aspartame | — | — | — | 4.8 | 1.5 | — |
| Glycyr- | — | 3.5 | — | — | — | — |
| rhizin | | | | | | |
| Neotame | 1.0 | — | — | 1.0 | — | 0.8 |
| Encap- | — | — | 1.0 | — | — | 1.0 |
| sulated | | | | | | |
| neotame | | | | | | |
| Color | 0.8 | 0.10 | 0.05 | 0.1 | 0.08 | 0.1 |
| Total Wt. | 1000.0 | 1000.0 | 1000.0 | 1000.0 | 1000.0 | 1000.0 |
| (grams) | | | | | | |

### Example 25

The bioadhesive of Example 4 is used and is added to 1.0% proteolytic enzyme to produce a product that adheres to buccal tissue.

### Example 26

Examples 3, 4, and 5 are used to make a multi-flavored, multi-layered and multi-colored bioadhesive product Example 27: Examples 14, 18, and 20 are used to make a multi-flavored and multi-layered bioadhesive film product that adheres specifically to the teeth of a consumer and contains a whitening agent.

### Example 28

Using Example 10, a bioadhesive product is produced that is shaped like the letter “U” and fits and adheres to the floor of the mouth of a consumer.

### Example 29

Using Example 9, a bioadhesive product is produced that is lemon-shaped.

### Example 30

Using Example 14, a bioadhesive product is produced adhering readily to the buccal tissue of the consumer to add green apple flavoring when consuming a beverage.

### Example 31

Using Example 20, a bioadhesive product is provided that includes a middle layer having bioadhesive properties, and the two outside layers consist of any of a variety of edible film compositions. This provides a confectionery product.

### Example 32

Examples 6 and 20 and an edible film composition are used to make a confectionery product.

### Example 33

Example 29 is incorporated into a hard-boiled candy.

### Example 34

Example 27 is modified so that the middle layer contains magnolia bark extract.

### Example 35

Example 24 is formulated into a multi-layered product; having layers with a thickness of 0.01 mm, 0.25 mm, and 0.15 mm, respectively.

### Example 36

Examples 10 and 14 are layered together. They are designed to be placed on a consumer's tongue. Example 10 contains glitter and Example 14 has orange and red stripes that leave coloring and glitter on the consumer's tongue.

### Example 37

Example 8 is designed into a product that adheres to any oral tissue, is placed in the mouth of a consumer at bedtime, and remains attached the oral tissue for up to 10 hours. The product provides anti-microbial agents, and therefore provides fresh breath to the consumer for the morning.

### Example 38

Example 22 is designed to adhere to tooth surfaces and contains and provides tooth whitening to the consumer.

### Example 39

Examples 19 and 23 are layered together and are adhered to a plastic non-edible film to make a “peel and pull” type product.

### Example 40

Example 24 is incorporated into a chewing gum product.

By way of example and not limitation, examples of preparing the bioadhesive products of the present invention will now be given.

Preparation Method for Bioadhesive or Bioerodible Components:

1). Blending:

a). Powdered materials (such as film formers, bioadhesive/bioerodible components) are blended together using a ribbon blender or similar device.

b). Flavors and flavor components/enhancers are blended together using mechanical agitation or other means.

2). Mixing:

a) The powdered mix is added to a mixing tank filled with the appropriate amount of water.

b) After the powdered mixture is mostly hydrated, the temperature is increased and softening agents, color or sweeteners may be added in succession as the temperature increases. The blend is maintained at an even temperature of 115-200° F.

3). Drying

a). Once the solution is well-blended, and reaches a temperature of approximately 180-200° F., the solution is coated onto a glass plate and a draw down bar is used to reach the desired thickness, and is cooled until dry.

By way of example and not limitation, examples of products and the uses thereof will now be given.

### Examples of Types of Bioadhesive Product

1. Breath Freshening Products—for freshening breath, these products can be used to combat the effects of certain foods, tobacco, halitosis, coffee, etc.

2. Dental Benefits

healthy mouth (kill germs that cause bad breath, gingivitis, caries)

tooth stain prevention and/or removal-whitening

strengthening (via remineralization, demin inhibition and/or neutralizing plaque acids to raise plaque pH above 5.5)

products designed to fit over the teeth to provide above effects during day or overnight

saliva stimulation to fight dry mouth 3. Breathe Free Products (nasal cooling); use of various cooling agents to impart cooling in the nasal tract.

4. Flavor and Sensations Products

different flavors in different product layers

different sensations (tingling, numbing, hot, cold, sweet, sour or combinations of these) in different product layers. Can combine sensations with different flavors for complex flavors

products with different flavors or sensations that can be placed simultaneously in different mouth locations

5. Alertness Products—product includes caffeine and other stimulants

6. Diet Aid Products—via flavors and other food approved agents, and those likely to obtain GRAS, to suppress desire to eat, burn calories, or other weight reduction effect.

It should be appreciated that various changes and modifications of the presently preferred embodiments described herein will be apparent to those skilled in the art. Such changes and modifications can be made without departing from the original spirit and scope of the present invention without diminishing its intended advantages. It is therefore intended that such changes and modifications be covered by the appended claims.

The invention is claimed as follows:

1. A consumable product comprising at least two layers, one of the two layers having bioadhesive characteristics.

2. The consumable product of claim 1 wherein one of the two layers comprises an edible thin film.

3. The consumable product of claim 1 including a third layer.

4. The consumable product of claim 1 comprising:
a first layer defining a top surface of the product, the second layer defining the middle layer of the product, and a third layer defining the bottom layer of the product.

5. The consumable product of claim 4 wherein the first layer is an edible film and the third layer having bioadhesive properties.

6. The consumable product of claim 4 wherein the first layer is an edible film and the second layer having bioadhesive properties.

7. The consumable product of claim 1 comprising a medicament.

8. The consumable product of claim 1 wherein one of the two layers comprises a chewing gum composition.

9. The consumable product of claim 1 wherein one of the two layers comprises a hard candy composition.

10. The consumable product of claim 1 wherein one of the two layers comprises a bioerodible disc.

11. The consumable product of claim 1 wherein one of the two layers comprises a chocolate containing composition.

12. The consumable product of claim 1 wherein one of the two layers comprises a soft candy composition.

13. The consumable product of claim 7 wherein the medicament is selected from the group consisting of pH control agent, tartar control, malodor control, breath freshening agents, caries control, enzymes, anti-plaque/anti-gingivitis agents, remineralization/demineralization agents, saliva stimulating agents, energizing agents, pharmaceutical agents, nutraceutical agents, vitamins, minerals, and combinations thereof.

14. The consumable product of claim 1 comprising a flavor chosen from the group consisting of essential oils, synthetic flavors and mixtures.

15. The consumable product of claim 14 wherein the flavor is an oil derived from plant and fruit oils selected from the group consisting of citrus oil, fruit essences, peppermint oil, spearmint oil, other mint oils, oil of wintergreen and anise.

16. The consumable product of claim 1 including a component selected from the group consisting of menthol, eucalyptol, thymol, methyl salicylate, licorice, and cinnamic aldehyde.

17. The consumable product of claim 1 including a backing covering at least one of the layers.

18. The consumable product of claim 17 wherein the layers are a different color than the backing.

19. The consumable product of claim 1 wherein the consumable product includes sides that are not parallel.

20. The consumable product of claim 1 wherein the product is shaped to adhere to an area of an oral cavity of a consumer.

21. The consumable product of claim 1 wherein at least one layer includes a surface that is textured.

22. The consumable product of claim 1 wherein at least one surface has a design.

23. The consumable product of claim 1 including an ingredient that creates an oral sensation in an oral cavity of the mouth of a consumer consuming the consumable product.

24. The consumable product of claim 1 wherein upon dissolution of the bioadhesive layer, the bioadhesive releases mucoadhesive micro beads that adhere to mucosal (surfaces for longer lasting oral sensations.

25. A delivery system that releases at least one of a flavor, sweetener, cooling agent, and active ingredient to a consumer consuming the delivery system comprising:
a first layer that comprises a thin film;
a second layer that comprises a water-soluble adhesive that adheres to a mucosal surface of the consumer; and
a third layer that releases at least one of the components chosen from the group consisting of flavor, sweetener, and cooling agent as it dissolves.

26. The delivery system of claim 25 wherein the first and third layers define an outer surface and the second layer is sandwiched therebetween.

27. The delivery system of claim 25 wherein the second layer defines an upper or lower outer surface.

28. The delivery system of claim 25 wherein the first layer releases one of the components chosen from the group consisting of flavor, sweetener, and cooling agents as it dissolves.

29. The delivery system of claim 25 wherein the second layer released one of the components chosen from the group consisting of flavor, sweetener, and cooling agents as it dissolves.

30. The delivery system of claim 25 wherein the active agent is a medicament selected from the group consisting of pH control agent, tartar control, malodor control, breath freshening agents, caries control, enzymes, anti-plaque/anti-gingivitis agents, remineralization/demineralization agents, saliva stimulating agents, energizing agents, pharmaceutical agents, nutraceutical agents, vitamins, minerals, and combinations thereof.

31. The delivery system of claim 25 comprising a flavor chosen from the group consisting of essential oils, synthetic flavors and mixtures.

32. The delivery system of claim 25 wherein the flavor is an oil derived from an oil selected from the group consisting of citrus oil, fruit essences, peppermint oil, spearmint oil, other mint oils, oil of wintergreen and anise.

33. The consumable product of claim 25 including a component selected from the group consisting of menthol, eucalyptol, thymol, licorice, and cinnamic aldehyde.

34. The delivery system of claim 25 wherein the bioadhesive layer is designed to adhere to the masticatory mucosa of the consumer.

35. The delivery system of claim 25 wherein the bioadhesive layer is designed to adhere to the specialized mucosa of the consumer.

36. The delivery system of claim 25 wherein the bioadhesive layer is designed to adhere to teeth of the consumer.

37. The delivery system of claim 25 wherein the bioadhesive layer is designed to adhere to the cheek of the consumer.

38. The delivery system of claim 25 wherein the bioadhesive layer is designed to adhere to the ventral tongue of the consumer.

39. The delivery system of claim 25 wherein the bioadhesive layer is designed to adhere to the gums surrounding the teeth of the consumer.

40. The delivery system of claim 25 wherein the bioadhesive layer is designed to adhere to the roof of the mouth of the consumer.

41. The delivery system of claim 25 wherein the bioadhesive layer is designed to adhere to specific regions on the tongue to stimulate certain tastes.

42. The delivery system of claim 25 wherein the bioadhesive layer comprises citric acid.

43. The delivery system of claim 25 wherein the bioadhesive layer is capable of being attached to a surface in the oral cavity of the consumer for at least five minutes.

44. The delivery system of claim 25 wherein the bioadhesive layer is capable of being attached to a surface in the oral cavity of the consumer for at least one hour.

45. The delivery system of claim 25 wherein the bioadhesive layer is capable of being attached to a surface in the oral cavity of the consumer for up to 10 hours.

46. The delivery system of claim 25 wherein the bioadhesive layer comprises at least one water-soluble polymer chosen from the group consisting of agar, alginic acid, alginate, amylose, high amylose starch, gum arabic, carrageenan, processed euchema seaweed, casein, carboxymethyl cellulose (CMC), carboxyvinyl copolymer, hydroxypropylcellulose (HPC), hydroxypropylmethylcellulose (HPMC), hydroxyethylcellulose (HEC), methyl cellulose, microcrystalline cellulose (MCC), sodium carboxymethyl cellulose, natural celluloses, chitin, chitosan, collagen, dextran, polydextran, elsinan, gelatin, gellan gum, guar gum, gelatin, ghatti gum, karaya gum, gluten, konjac, levan, locust bean gum, maltodextrin, methylmethacrulate copolymer, oat gum, pectin, low methoxy pectin, polyethylene glycol, polylysine, polybrene, polyvinylpyrrolidone, polyvinyl alcohol, polyacrylic acid, propylene glycol, protein, pullulan, starch, modified starches, soy protein, tara gum, tamarind gum, tragacanth, whey protein, xanthan gum, and zein.

47. A product that is designed to adhere to a portion of the mouth of a consumer comprising:
an outer surface that is designed to rapidly dissolve and expose a bioerodible surface that releases components chosen from the group consisting of flavor, sweetener, cooling agent, and active ingredient.

48. The product of claim 47 wherein the outer surface is an edible thin film.

49. The product of claim 47 wherein the outer surface releases a flavor into the mouth of the consumer.

50. The product of claim 47 comprising a medicament.

51. A method for producing a confectionery product comprising the steps of manufacturing a multilayered product including a top and bottom surface that are not bioadhesives and an inner layer that is a bioadhesive.

52. The method of claim 51 comprising the steps of manufacturing the product so that the first layer comprises an edible thin film.

53. The method of claim 51 comprising the steps of manufacturing the product so that the first and third layers comprise edible thin films.

54. The method of claim 51 comprising the steps of manufacturing the product so that the first layer is a bioerodible disc.

55. The method of claim 51 comprising the steps of manufacturing the product so that the first layer is an edible thin film and the third layer comprises a bioerodible disc.

56. The method of claim 51 comprises the steps of manufacturing the product so that the bioadhesive layer comprises at least one water-soluble polymer and at least one water-insoluble polymer.

57. The method of claim 51 comprises the steps of manufacturing the product so that the product comprises a medicament.

58. The method of claim 57 comprising the steps of manufacturing the product so that the medicament is selected from the group consisting of pH control agent, tartar control, malodor control, breath freshening agents, caries control, enzymes, anti-plaque/anti-gingivitis agents, remineralization/demineralization agents, saliva stimulating agents, energizing agents, pharmaceutical agents, nutraceutical agents, vitamins, minerals, and combinations thereof.

59. The method of claim 51 comprising the steps of manufacturing the product so that the product comprises a flavor chosen from the group consisting of essential oils, synthetic flavors and mixtures.

60. The method of claim 59 wherein the flavor is an oil derived from plant and fruit oils selected from the group consisting of citrus oil, fruit essences, peppermint oil, spearmint oil, other mint oils, oil of wintergreen and anise.

61. The method of claim 51 wherein the product includes a component selected from the group consisting of menthol, eucalyptol, thymol, methyl salicylate, licorice, and cinnamic aldehyde.

62. The method of claim 51 comprising the steps of adding a backing to the first or the third surface.

63. The method of claim 51 wherein the layers are a different color than the backing portion.

64. The method of claim 51 comprising the step of manufacturing the product so that it includes sides that are not parallel.

65. The method of claim 51 including the step of manufacturing at least one layer to include a surface that is textured.

66. The method of claim 51 comprising the step of adding to at least one surface a design.

67. A method of producing a product comprising:
manufacturing a product including at least a portion that has bioadhesive characteristics and a flavor that is distinctive of a confectionery product that was marketed prior to the product.

68. The method of claim 67 comprises the steps of manufacturing the product so that the product comprises a medicament.

69. The method of claim 68 comprising the steps of manufacturing the product so that the medicament is selected from the group consisting of pH control agent, tartar control, malodor control, breath freshening agents, caries control, enzymes, anti-plaque/anti-gingivitis agents, remineralization/demineralization agents, saliva stimulating agents, energizing agents, pharmaceutical agents, nutraceutical agents, vitamins, minerals, and combinations thereof.

70. The method of claim 67 comprises the steps of manufacturing the product so that the product comprises a medicament.

71. The method of claim 67 wherein the product includes a component selected from the group consisting of menthol, eucalyptol, thymol, methyl salicylate, licorice, and cinnamic aldehyde.

72. A method for developing new bioadhesive products comprising the steps of:
causing at least one entity to enter into a development effort with a second entity to produce a new bioadhesive product wherein one of the entities possesses expertise with respect to bioadhesive products and another entity provides expertise with respect to confectionery products; and
selling a product that is developed pursuant to the development effort.

73. The method of claim 72 wherein each of the entities are companies.

74. The method of claim 72 wherein one of the entities sells products including medicaments.

75. The method of claim 72 wherein one of the entities sells chewing gum products.

76. The method of claim 72 wherein the step of causing entities to enter into a development effort includes the step of entering into a contract.

77. The method of claim 72 including the step of cross-licensing technology to develop the bioadhesive product.

78. A method of generating revenue comprising the steps of:
selling products comprising an outer surface that does not have bioadhesive characteristics and an inner layer having bioadhesive characteristics, the product providing to the consumer a characteristic chosen from the group consisting of breath freshening, dental benefits, nasal cooling, alertness, and diet aid.

79. A method of generating revenue comprising the step of developing a product that includes a bioadhesive layer, at least one flavor chosen from the group consisting of peppermint, spearmint, and cinnamon, and a sufficient amount of an active ingredient so that the product provides breath freshening to a consumer.

80. The method of claim 79 including the step of selling the product.

81. A method of generating revenue comprising the step of developing a product that includes a bioadhesive layer, at least one flavor chosen from the group consisting of peppermint, spearmint, and cinnamon, and a sufficient amount of an active ingredient so that the product provides dental benefits to a consumer.

82. The method of claim 81 including the step of selling the product.

83. A method of generating revenue comprising the step of developing a product that includes a bioadhesive layer, at least one flavor chosen from the group consisting of peppermint, spearmint, and cinnamon, and a therapeutically effective amount of at least one medicament to treat a disease of a consumer.

84. The method of claim 83 including the step of selling the product.

85. The method of claim 83 wherein the medicament is selected from the group consisting of pH control agent, tartar control, malodor control, breath freshening agents, caries control, enzymes, anti-plaque/anti-gingivitis agents, remineralization/demineralization agents, saliva stimulating agents, energizing agents, pharmaceutical agents, nutraceutical agents, vitamins, minerals, and combinations thereof.

86. A delivery system that releases at least one of a flavor, sweetener, cooling agent, and active ingredient to a consumer consuming the delivery system comprising:
a first layer comprising a bioadhesive composition that adheres to a mucosal surface of a consumer;
a second layer comprising a water-soluble bioerodible composition that releases one of the components chosen from the group consisting of flavor, sweetener, and cooling agent as it dissolves; and
a third layer that comprises an edible film.

87. The delivery systems of claim 86 wherein upon the dissolution of the bioerodible layer, the bioerodible layer releases mucoadhesive beads that adhere to mucosal surfaces for longer lasting oral sensations.

88. The delivery system of claim 86 wherein the bioerodible composition comprises at least one water-soluble polymer chosen from the group consisting of agar, alginic acid, alginate, amylose, high amylose starch, gum arabic, carrageenan, processed euchema seaweed, casein, carboxymethyl cellulose (CMC), carboxyvinyl copolymer, hydroxypropylcellulose (HPC), hydroxypropylmethylcellulose (HPMC), hydroxyethylcellulose (HEC), methyl cellulose, microcrystalline cellulose (MCC0, sodium carboxymethyl cellulose, natural celluloses, chitin, chitosan, collagen, curdlan, dextran, polydextran, elsinan, gelatin, gellan gum, guar gum, ghatti gum, karaya gum, gluten, konjac, levan, locust bean gum, maltodextrin, methylmethacrylate copolymer, oat gum, pectin, low methoxy pectin, polyethylene glycol, polylysine, polybrene, polyvinylpyrrolidone, polyvinyl alcohol, polyacrylic acid, propylene glycol, protein, pullulan, starch, modified starches, soy protein, tara gum, tamarind gum, tragacanth, whey protein, xanthan gum, and zein.

89. The delivery system of claim 88 wherein the water-soluble polymer comprises 1% to about 60% by wet weights of the bioerodible composition.

90. The delivery system of claim 86 wherein the bioadhesive layer releases one of the components chosen from the group consisting of flavor, sweetener, and cooling agent as it dissolves.

91. The delivery system of claim 86 comprising a fourth layer.

92. The delivery system of claim 91 wherein the fourth layer is chosen from the group consisting of edible films, bioerodible compositions, hard candy, soft candy, chewing gum, and chocolate.
